(19) United States
(12) Patent Application Publication
(10) Pub. No.: US 2006/0270005 A1
(43) Pub. Date: Nov. 30, 2006

(54) Genomic library of cyanophage s-2l and functional analysis

(75) Inventors: Philippe Marliere, Paris (FR); Pierre-Alexandre Kaminski, Paris (FR); Frederique Galisson, Finhaul (CH); Madaleine Bouzon, Meudon (FR); Sylvie Pochet, Paris (FR); Jean Weissenbach, Paris (FR); William Saurin, Paris (FR); Catherine Robert, Evry (FR); Virginie VIco, Evry (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignees: INSTITUT PASTEUR, Paris (FR); CENTRE NATIONAL DE LA RECHERCHE SCIENTIFIQUE, Paris (FR); GENOSCOPE- CENTRE NATIONAL DE SEQUENCAGE, Evry (FR)

(21) Appl. No.: 10/510,953

(22) PCT Filed: Apr. 28, 2003
(86) PCT No.: PCT/FR03/01328

§ 371 Date: Dec. 2, 2005

(30) Foreign Application Priority Data
Apr. 30, 2002 (FR) 02/05424

(51) Int. Cl. C12P 19/34; C12N 9/22; C07H 21/04; C12N 7/00; C12N 15/74; C07K 14/005

(52) U.S. Cl. 435/91.2; 536/23.72; 435/472; 435/252.3; 435/235.1; 530/350; 435/199

(57) ABSTRACT

The invention relates to the genome sequence and the nucleotide sequences coding for polypeptides of cyanophage S-2L. According to the invention, the polypeptides include, but are not limited to, polypeptides involved in the synthesis, transcription and replication of purine bases. In particular, determining the genome of cyanophage S-2L is useful for supplying genes which, expressed in recombinant bacteria, enable the synthesis of DNA monomers incorporating base D (2,6 diaminopurine instead of base A (adenine), thereby producing chemically-remodelled nucleic acids in the bacteria.

A subject of the present invention is the gene sequence and nucleotide sequences coding for polypeptides of cyanophage S-2L. The polypeptides described in the present invention are, in a non-limitative way, polypeptides involved in the synthesis, transcription and replication of purine bases. In particular, the determination of the genome of cyanophage S-2L is a useful tool for producing genes, which, expressed in recombinant bacteria, allow the synthesis of DNA monomers incorporating the D-base (2,6 diaminopurine) instead of the A-base (adenine) and thus the production of chemically remodelled nucleic acids in the bacteria.

The invention also relates to the use of the gene sequence and/or of the nucleotide and/or polypeptide sequences described in the present invention for the analysis of the expression of genes.

The two main nucleic acids DNA and RNA are polymers of nucleotides which are made up of a purine or pyrimidine base linked to a sugar with 5 carbons (deoxyribose in the case of DNA, ribose in RNA) via an N-glycosidic bond and an esterified phosphate with the hydroxyl carbon group situated in position 5′ of the sugar: RNA and DNA contain four types of nucleotides which are distinguished by their bases: adenine (A), guanine (G), cytosine (C) and uracil (U) for RNA; 5-methyluracil, i.e. thymine (T), replacing uracil in DNA.

Amongst the possible chemical alterations of DNA and of RNA only modifications of the bases and not of the sugar were observed. By contrast with DNA no modified RNA has been able to be replicated until now.

Modified bases are observed in the DNA of all organisms, and can be involved in phenomena of regulation of gene expression (5). Except in bacteriophages, the DNA modifications known until now are produced by post-replicative enzymatic reactions, a DNA duplex of which is the substrate.

By contrast, during infection with certain bacteriophages, the DNA modifications known until now are produced by prereplicative enzymatic reactions, a nucleotide of which is the substrate, in order to lead to a non-canonical triphosphate deoxynucleoside. Among the known modifications the following entities are mentioned: dUTP, 5-hydroxymethyl-dUTP, 5-dihydroxypentyl-dUTP, 5-hydroxymethyl-dCTP. Another entity is strongly suspected: 5-methyl-dCTP (11). The emergence of the modified bases in the bacteriophages is generally interpreted as a counter-measure to the bacteria restriction systems (11).

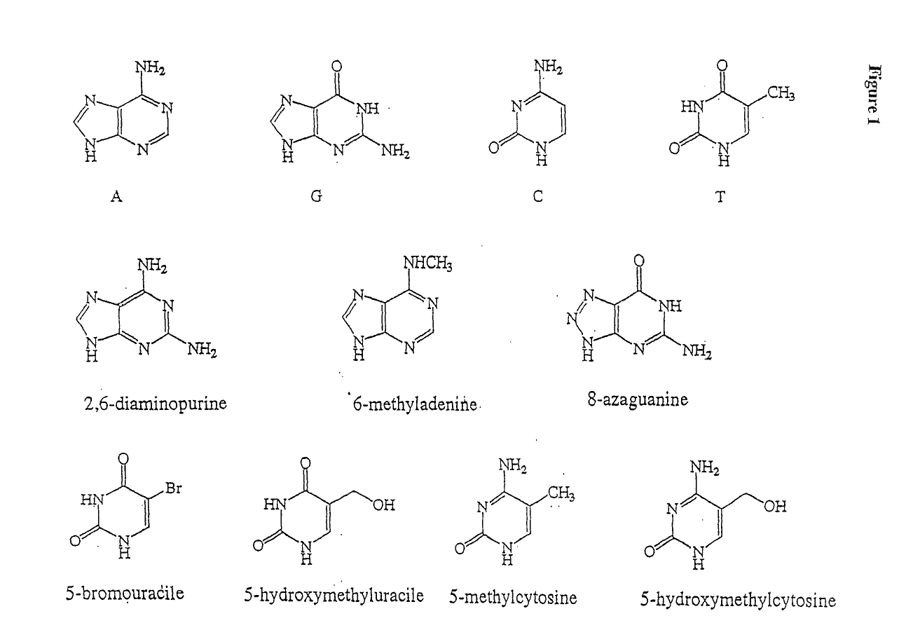

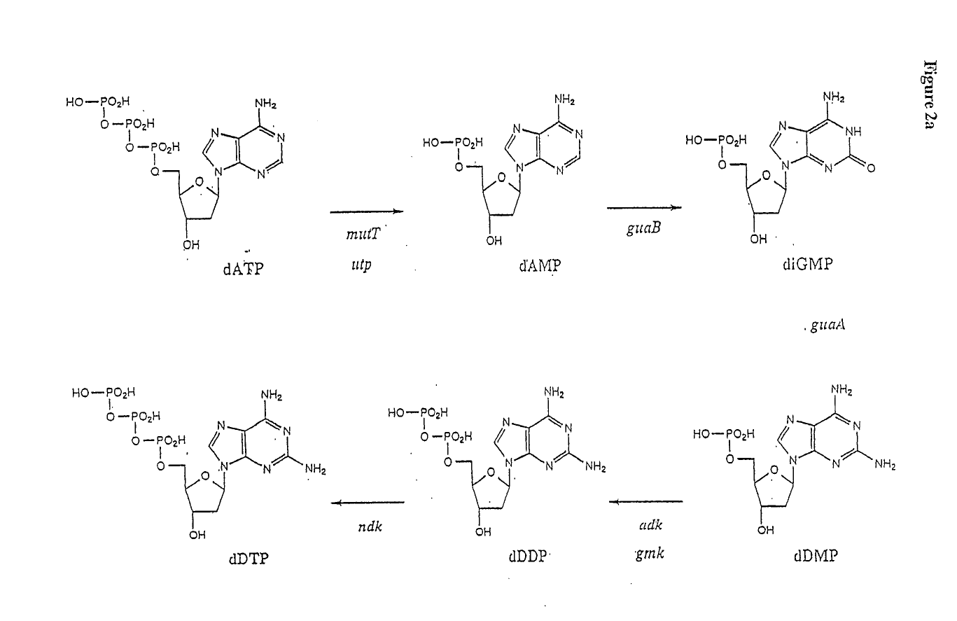

A few examples of modified bases are shown in FIG. 1.

Bromouracil or 8-azaguanine are synthetic analogues of the natural bases thymine and guanine. These analogues are converted into triphosphate nucleotides by the protection pathways of the purines or pyrimidines and are then incorporated into the DNA. 6-methyladenine and 5-methylcytosine are the most frequently encountered modified bases. The methylated nucleotides are not incorporated as such in the DNA but are the product of the action of specific DNA methyltransferases. These enzymes transfer the methyl group of S-adenosylmethionine to the adenine or cytosine, after the replication of the DNA. In the prokaryotes the main role of DNA methylation is the degradation of the foreign DNA. In the eukaryotes DNA methylation influences the regulation of gene expression and cell differentiation.

In certain T-type phages such as bacteriophage T4, the cytosine is systematically replaced by 5-hydroxymethylcytosine. This substitution requires on the one hand a biosynthesis route of hydroxymethyl deoxycytidine triphosphate (HMdCTP) as well as enzymes allowing the exclusion of the normal base.

The biosynthesis route of the HMC-DNA involves a hydroxymethylase which converts the dCMP into hydroxymethyl dCMP, a nucleoside monophosphate kinase which phosphorylates the HM dCMP in order to produce diphosphate, precursor of HM dCTP which is then incorporated into the DNA polymerase then glycosylated by a glycosyltransferase.

The exclusion of the cytosine involves on the one hand specific endonucleases of DNA containing this base and a dCDPase-dCTPase which converts the corresponding nucleotides into dCMP which is then the substrate of the dCMP hydroxymethylase and dCMP deaminase. The dCMP deaminase generates the dUMP precursor of dTMP.

By means of a mechanism similar to that described above, the thymine is replaced by 5-hydroxymethyluracil (phages SPOL and Φe) or uracil (phage PBS2) in several Bacillus subtilis phages (Warren, 1980; Komberg and Baker,1991).

Other phages such as SP15 or ΦW14 have a DNA whose thymine was replaced by 5-dihydroxypentyluracil and α-putrescinylthymine. However, this replacement is only partial and seems to be due to post-replicative modifications.

In the case of S-2L, the synthesis route of the D-base is not yet completely established and the post-replicative modification of adenine to diaminopurine cannot be totally avoided. However, the biosynthesis of the non-canonical dDTP monomer appears to be significantly more likely, given the fact that the replacement of A with D in the DNA of S-2L is total and not substantial (7,8), as is the case for the post-replicative modifications of hydroxymethyl-U to putrescine-T in the ΦW14 phage. Moreover, the modification of A to D in situ would require the rupture of the hydrogen bonds of the DNA duplexes and, being difficult to carry out in one chemical stage, would introduce mutagen lesions if this process were interrupted.

The Cyanophage S-2L

The cyanophage S-2L was isolated from water samples taken in the Leningrad region. This phage is capable of lysing a relatively restricted number of Synechococcus: sp. 698.58 and PCC6907. From a morphological point of view it is composed of an icosahedral head and a flexible non-contractile tail. S-2L belongs to a family whose other member could be the SM-2 phage which is morphologically similar (Fox et al. 1976).

The DNA of the S-2L phage is linear and double stranded with a size of 42 kb composed of 70% G:C and 30% of a pair equivalent to A:T in which the adenine has been replaced by 2,6-diaminopurine (D). This replacement is total and no other base has been able to be identified (Kimos et al., 1977; Khudyokov et al., 1978). As has been seen previously, only total replacements of pyrimidine bases have been reported, S-2L is the only case for a purine base to date.

As in the G:C pairs three hydrogen bonds are formed between the purine and the pyrimidine of the D:T pair which gives the DNA a greater stability.

The presence of the D-base in the DNA of S-2L causes a resistance to digestion by restriction endonucleases possessing an A in their recognition site (the restriction enzyme TaqI being the only exception). However, the D:T pair seems to be recognized as a G:C pair by the restriction enzymes cleaving the sequences rich in G:C such as SmaI (Szekeres and Matveyev, A. V., 1978).

Given that the A-base is totally and not just mostly replaced by D, it is very likely that the genome of the S-2L phage codes for at least one biosynthesis route of the D-base.

With regard to the prior art, the study of the cyanophage S-2L requires new approaches, in particular genetic ones, in order to improve the comprehension of the different metabolic routes of this organism.

Thus, an object of the present invention is to disclose the complete sequence of the genome of the cyanophage S-2L and of all the genes contained in said genome.

In fact, knowledge of the genome of this organism allows better definition of the interactions between the different genes, the different proteins, and, the different metabolic routes. In fact, in contrast to the disclosure of isolated sequences, the complete gene sequence of an organism forms a whole, making it possible immediately to obtain all the information necessary for this organism to grow and function.

The invention is in particular aimed at sequencing the genome of the S-2L phage, so as to obtain a pool of genes which, once propagated in isolation and expressed under control in recombinant bacteria, are intended in particular to form by biotechnological route new monomers of DNA and to produce, or replicate, chemically remodelled nucleic acids in bacteria.

The invention is also aimed at using nucleotide sequences obtained for the identification of the metabolic routes leading to the production of the D-bases.

The invention is also aimed at the enzymatic production of analogues of deoxynucleosides which are very useful in particular in chemotherapy for AIDS.

The invention is also aimed at expressing in a S2L cyanophage host nucleic acids coding for proteins involved in the metabolism of the D-bases.

Thus the invention is also aimed at obtaining S-2L genes which, propagated individually in E. coli and expressed under strict transcriptional control, allow testing of the hypotheses concerning their function in the metabolism of nucleotides, replication and transcription.

To achieve the various technical results sought, according to a first aspect the invention relates to a nucleotide sequence of cyanophage S-2L corresponding to SEQ ID No. 1.

The present invention also relates to a nucleotide sequence of cyanophage S-2L chosen from:
a) a nucleotide sequence comprising at least 80%, 85%, 90%, 95% or 98% identity with SEQ ID No. 1;
b) a nucleotide sequence hybridizing under high stringency conditions with SEQ ID No. 1;
c) a nucleotide sequence which complements SEQ ID No. 1 or which complements a nucleotide sequence as defined in a), or b), or a nucleotide sequence of the corresponding RNA;
d) a nucleotide sequence of a representative fragment of SEQ ID No. 1, or of a representative fragment of a nucleotide sequence as defined in a), b) or c);

e) a nucleotide sequence comprising a sequence as defined in a), b), c) or d); and
f) a nucleotide sequence modified from a nucleotide sequence as defined in a), b), c), d) or e).

More particularly, a subject of the present invention is nucleotide sequences characterized in that they are from SEQ ID No. 1 and in that they code for polypeptides chosen from the sequences SEQ ID No. 2 to SEQ ID No. 527 or a biologically active fragment of these polypeptides.

Moreover, the invention also relates to the nucleotide sequences characterized in that they comprise a nucleotide sequence chosen from:
a) a nucleotide sequence from SEQ ID No. 1 and coding for a polypeptide chosen from the sequences from SEQ ID No. 2 to SEQ ID No. 527.
b) a nucleotide sequence comprising at least 80%, 85%, 90%, 95% or 98% identity with a nucleotide sequence according to a);
c) a nucleotide sequence hybridizing under high stringency conditions with a nucleotide sequence according to a) or b);
d) a nucleotide sequence which is complementary or from RNA corresponding to a sequence as defined in a), b) or c);
e) a nucleotide sequence of a representative fragment of a sequence as defined in a), b), c) or d); and
f) a nucleotide sequence modified from a sequence as defined in a), b), c), d) or e),

Preferably the invention relates to a nucleotide sequence characterized in that it codes for a polypeptide chosen from:

a) the polypeptides of the cyanophage S-2L of sequences SEQ ID No. 2 to SEQ ID No. 527;

b) preferably the 54 polypeptides mentioned in Table 1 namely: SEQ ID No. 14, 18, 26, 68, 86, 92, 105, 109, 134, 142, 143, 148, 152, 169, 175, 187, 208, 211, 234, 246, 250, 257, 264, 286, 298, 316, 332, 342, 347, 348, 351, 355, 364, 365, 369, 370, 392, 395, 406, 418, 422, 425, 429, 432, 433, 454, 464, 466, 472, 484, 489, 494, 500;

c) preferably also the 14 polypeptides of the cyanophage S-2L shown in Table 1 as having a significant homology namely the sequences SEQ ID No. 86, 92, 152, 175, 234, 257, 298, 316, 395, 406, 425, 484;

d) the polypeptides having at least 80% preferably 85%, 90%, 95% and 98% identity with a polypeptide from a), b), c);

e) the biologically active fragments of the polypeptides from a), b), c), d) f) the polypeptides modified from a), b), c), d), e).

The invention also relates to a nucleotide sequence characterized in that it comprises a nucleotide sequence chosen from:
a) a nucleotide sequence as defined above;
b) a nucleotide sequence comprising at least 80% identity with a nucleotide sequence from a);
c) a nucleotide sequence hybridizing under high stringency conditions with a nucleotide sequence from a) or b);
d) a nucleotide sequence which is complementary or from RNA corresponding to a sequence as defined in a), b) or c);
e) a nucleotide sequence of a representative fragment of a sequence as defined in a), b), c) or d); and
f) a nucleotide sequence modified from a sequence as defined in a), b), c), d) or e).

By nucleic acid, nucleic or nucleic acid sequence, polynucleotide, oligonucleotide, polynucleotide sequence, nucleotide sequence, terms which are used indiscriminately in the present description, is meant a specific sequence of nucleotides, modified or not modified, allowing the definition of a fragment or a region of a nucleic acid, comprising or not comprising unnatural nucleotides, and able to correspond to a double strand DNA, a single strand DNA as well as the transcription products of said DNAs. Thus, the nucleic sequences according to the invention also include the PNAs (Peptide Nucleic Acid), or analogues.

It must be understood that the present invention does not relate to nucleotide sequences in their natural chromosomal environment, i.e. in the natural state. It concerns sequences which have been isolated and/or purified, i.e. that have been sampled directly or indirectly, for example by copying, their environment having been at least partially modified. It thus also designates the nucleic acids obtained by chemical synthesis.

By “identity percentage” between two nucleic acid or amino acid sequences in the context of the present invention, is meant a percentage of nucleotides or amino acid residues which are identical in the two sequences to be compared, obtained after the best alignment, this percentage being purely statistical and the differences between the two sequences being randomly distributed and over all of their length. By “best alignment” or “optimal alignment” is meant the alignment in which the identity percentage as determined below is highest. The comparison of sequences between two nucleic acid or amino acid sequences are traditionally carried out by comparing these sequences after having aligned them in an optimal manner, said comparison being carried out by segment or by “window of comparison” in order to identify and compare the local regions with sequence similarity. The optimal alignment of the sequences for the comparison can be achieved, as well as manually, using the local homology algorithm of Smith and Waterman (1981, Ad. App. Math. 2: 482), the local homology algorithm of Neddleman and Wunsch (1970, J. Mol. Biol. 48: 443), the similarity search method of Pearson and Lipman (1988, Proc. Natl. Acad. Sci. USA 85: 2444), or information technology software using these algorithms (GAP, BESTFIT, BLAST P, BLAST N, FASTA and TFASTA in the Wisconsin Genetics Software Package, Genetics Computer Group, 575 Science Dr., Madison, Wis.). In order to obtain the optimal alignment, the BLAST program is preferably used, with the BLOSUM 62 matrix. The PAM or PAM250 matrixes can also be used.

The identity percentage between two nucleic acid or amino acid sequences is determined by comparing these two sequences aligned in an optimal manner in which the nucleic acid or amino acid sequence to be compared can comprise additions or deletions in relation to the reference sequence for an optimal alignment between these two sequences. The identity percentage is calculated by determining the number of identical positions in which the nucleotide or the amino acid residue is identical in the two sequences, by dividing this number of identical positions by the total number of positions compared and multiplying the result obtained by 100 in order to obtain the identity percentage between these two sequences.

By nucleic sequences having an identity percentage of at least 80%, preferably 85% or 90%, particularly preferably 95% or even 98%, after optimal alignment with a reference sequence, is meant nucleic sequences having, in relation to the reference nucleic sequence, certain modifications such as in particular a deletion, truncation, extension, a chimeric fusion and/or a substitution, in particular punctual, and the nucleic sequence of which has at least 80%, preferably 85%, 90%, 95% or 98%, identity after optimal alignment with the reference nucleic sequence. These are preferably sequences the complementary sequences of which are able to hybridize specifically with the reference sequences. Preferably, the specific or high stringency hybridization conditions are such that they ensure at least 80%, preferably 85%, 90%, 95% or 98% identity after optimal alignment between one of the two sequences and the complementary sequence of the other.

A hybridization under high stringency conditions means that the temperature and ionic force conditions are chosen so that they allow the hybridization to be maintained between two complementary DNA fragments. By way of example, high stringency conditions of the hybridization stage for the purpose of defining the polynucleotide fragments described above, are advantageously as follows.

The DNA-DNA or DNA-RNA hybridization is carried out in two stages:(l) prehybridization at 42° C. for 3 hours in phosphate buffer (20 mM, pH 7.5) containing 5×SSC (1×SSC corresponds to a 0.15 M NaCl +0.015 M sodium citrate solution), 50% formamide, 7% sodium dodecyl sulphate (SDS), 10× Denhardt's, 5% dextran sulphate and 1% of salmon sperm DNA; (2) standard hybridization for 20 hours at a temperature dependent on the size of the probe (i.e.: 42° C., for a probe of size >100 nucleotides) followed by 2 20-minute washings at 20° C. in 2×SSC +2% SDS, 1×20-minute washing at 20° C. in 0.1×SSC +0.1% SDS. The last washing is carried out in 0.1×SSC +0.1% SDS for 30 minutes at 60° C. for a probe of size >100 nucleotides. The high stringency hybridization conditions described above for a polynucleotide of a defined size, can be adapted by a person skilled in the art for oligonucleotides of greater or smaller size, according to the teaching of Sambrook et al., (1989, Molecular cloning: a laboratory manual.2nd Ed. Cold Spring Harbor).

Moreover, by representative fragment of sequences according to the invention, is meant any nucleotide fragment having at least 15 nucleotides, preferably at least 20, 30, 75, 150, 300 and 450 consecutive nucleotides of the sequence from which it originates. In particular a nucleic sequence coding for a biologically active fragment of a polypeptide, such as defined subsequently, in particular of a polypeptide of sequence SEQ ID No. 2 to 527 is meant.

By representative fragment, is also meant the intergene sequences, and in particular the nucleotide sequences carrying the regulation signals (promoters, terminators, or enhancers etc.).

Amongst said representative fragments, those are preferred which have the nucleotide sequences corresponding to open reading frames, called ORFs (Open Reading Frame) sequences, generally comprised between a start codon and a stop codon, or between two stop codons, and coding for polypeptides, preferably of at least 30 amino acids, such as for example, non-limitatively, the ORFs sequences which are described below.

The numbering of nucleotide ORFs sequences which is used subsequently in the present description corresponds to the numbering of the amino acid sequences of the proteins coded by said ORFs.

Thus, the nucleotide sequences ORF2, ORF3 etc.,ORF526 and ORF527 code respectively for the proteins of amino acid sequences SEQ ID No. 2, SEQ ID No. 3 etc., SEQ ID No. 526 and SEQ ID No. 527 which appear in the list of sequences of the present invention. The detailed nucleotide sequences of the ORF2, ORF3 etc., ORF526 and ORF527 sequences are determined by their respective position on the gene sequence SEQ ID No. 1 of the cyanophage S-2L. Table 1 gives the coordinates of 54 preferred ORFs in relation to the nucleotide sequence SEQ ID No. 1, giving the starting nucleotide, the nucleotide at the end of the ORF, and the reading frame +1,2,3 or −1,2,3 as explained below. The sequence listing shows the reading frame for each of the 526 ORFs identified, numbered ORF2 to ORF527. For perfect concordance between the numbering of the ORF and the SEQ ID in the sequence listing, it was decided to start the ORF at number 2 (thus there is no ORF 1). It is understood that sequence SEQ ID No. 1 is a DNA strand in the 5′-3′ orientation, sequence SEQ ID No. 2 is a protein sequence coded by ORF No. 2. A “positive” frame of +1 corresponds to the reading frame called +1 beginning at nucleotide nt 3 of SEQ ID No. 1 (1^{st} codon of ORF2 situated on this reading frame and beginning at nt 9 of SEQ ID No. 1: TCG which corresponds to serine S; 2^{nd} codon of ORF 2 according to this frame: GAG which corresponds to glutamic acid E). A frame +2 corresponds to the reading frame called +2 beginning at the nucleotide nt 1 of SEQ ID No. 1 (1^{st} codon of ORF 4 situated on this reading frame and beginning at the nt 10 of SEQ ID No. 1: CGG which corresponds to arginine R; 2 codon of ORF 4 according to this frame: AGG which corresponds to arginine R). A frame +3 corresponds to the reading frame called +3 beginning at nucleotide nt 2 of SEQ ID No. 1 (1^{st} codon of ORF 5 situated on this reading frame and beginning at the nt 35 of SEQ ID No. 1: CGT which corresponds to arginine R; 2^{nd} codon of ORF 5 according to this frame: TCA which corresponds to serine S).

Thus ORF 2 begins at nt No. 9 of SEQ ID No. 1 (i.e. the T-base ) and ends at nt No. 515 (i.e. G-base). ORF 4 begins at nt No. 10 of SEQ ID No.1 (i.e. the T-base) and ends at nt No. 342 (i.e. the G-base). ORF 5 begins at nt No. 35 of SEQ ID No. 1 (i.e. the C-base) and ends at nt No. 280 (i.e. the A-base).

Conversely, a negative frame corresponds to the antiparallel complementary strand of the positive strand. For example for an ATG sequence on the positive strand in the direction 5′-3′, the sequence on the complementary TAC strand is read CAT. For example for ORF 3 (nt 9 to nt 791), the complementary strand of nucleotides 782 to 791 (CCT CGA TAG) is (GGA GCT ATC) reading in negative direction CTA TCG AGG which corresponds respectively to the amino acids L, S, R.

The representative fragments according to the invention can be obtained for example by specific amplification such as PCR or after digestion by appropriate restriction enzymes of nucleotide sequences according to the invention, this method being described in particular in the work of Sambrook et al. Said representative fragments can also be obtained by chemical synthesis when their size is not too large, according to methods which are well known to a person skilled in the art.

Amongst the sequences containing sequences of the invention, or representative fragments, the sequences which are naturally surrounded by sequences which have at least 80%, 85%, 90%, 95% or 98% identity with the sequences according to the invention are also implied.

By modified nucleotide sequence, is meant any nucleotide sequence obtained by mutagenesis according to techniques well known to a person skilled in the art, and comprising modifications in relation to the normal sequences, for example mutations in the sequences which regulate and/or promote polypeptide expression, in particular leading to a modification of the level of expression or activity of said polypeptide.

By modified nucleotide sequence, is also meant any nucleotide sequence coding for a modified polypeptide such as defined below.

The present invention provides all of the nucleotide and polypeptide sequences of the cyanophage S-2L genome. Moreover, it is a subject of the present invention to disclose the functions of these genes and proteins.

The genes described in the invention were isolated on fragments of DNA using primers taken from the cyanophage S-2L sequence.

Preferably, the invention relates to a nucleotide sequence characterized in that it codes for a polypeptide of cyanophage S-2L or one of its representative fragments involved in the metabolism of nucleotides, purines, pyrimidines or nucleosides. In this text, the term “representative fragment” for a peptide means a biologically active fragment of this peptide (having an activity of at least 10, 20, 50, 100% of the activity obtained with this peptide).

In particular the invention relates to a nucleotide sequence characterized in that it codes for a polypeptide of cyanophage S-2L or one of its representative fragments involved in the metabolism of D-base nucleotides, in particular a peptide of sequence SEQ ID No. 175 or one of its representative fragments.

Preferably, the invention relates to a nucleotide sequence characterized in that it codes for a polypeptide of cyanophage S-2L or one of its representative fragments involved in the replication process, in particular a peptide of sequence SEQ ID No. 14, 18, 142, 355, 429, 454 or one of their representative fragments.

Preferably, the invention relates to a nucleotide sequence characterized in that it codes for an envelope, in particular a capsid polypeptide, of cyanophage S-2L or one of its representative fragments, in particular a peptide of sequence SEQ ID No. 169, 316, 351, 392, 395, 406, 422, 425 or one of their representative fragments.

Preferably, the invention relates to a nucleotide sequence according to the invention characterized in that it codes for a polypeptide of cyanophage S-2L or one of its fragments involved in the rerouting of the cell machinery.

Preferably, the invention relates to a nucleotide sequence according to the invention characterized in that it codes for a polypeptide of cyanophage S-2L or one of its representative fragments involved in the transcription process, in particular a peptide of sequence SEQ ID No. 92, 143, 187, 234 or one of their representative fragments.

Preferably, the invention relates to a nucleotide sequence according to the invention characterized in that it codes for a polypeptide of cyanophage S-2L or one of its representative fragments involved in the viral virulence process, in particular a peptide of sequence SEQ ID No. 257 or a representative fragment.

Preferably, the invention relates to a nucleotide sequence according to the invention characterized in that it codes for a polypeptide of cyanophage S-2L or one of its representative fragments involved in the functions relating to transposons in particular a peptide of sequence SEQ ID No. 208 or one of its representative fragments.

The representative fragments of nucleotide sequences according to the invention can also be probes or primers, which can be used in processes of detection, identification, assay or amplification of nucleic sequences.

A probe or primer is defined, in the context of the invention, as being a single strand nucleic acid fragment or a denatured double strand fragment comprising for example from 12 bases with several kb, in particular from 15 to several hundred bases, preferably from 15 to 50 or 100 bases, and having a hybridization specificity under determined conditions in order to form a hybridization complex with a target nucleic acid.

The probes and primers according to the invention can be marked directly or indirectly with a radioactive or non-radioactive compound by methods well known to a person skilled in the art, in order to obtain a detectable and/or quantifiable signal.

The unmarked sequences of polynucleotides according to the invention can be used directly as a probe or primer.

The sequences are generally marked in order to obtain sequences which can be used for many applications. The marking of the primers or probes according to the invention is carried out with radioactive elements or with non-radioactive molecules.

Among the radioactive isotopes used, ³²P, ³³P, ³⁵S, ³H or ¹²⁵I, can be mentioned. The non-radioactive entities are selected from the ligands such as biotin, avidin, streptavidin, dioxigenin, haptens, colourants, luminescent agents such as radioluminescent, chemiluminescent, bioluminescent, fluorescent, phosphorescent agents.

The polynucleotides according to the invention can thus be used as primer and/or probe in processes using in particular the PCR technique (polymerase chain amnplification) (Rolfs et al.,1991, Berlin: Springer-Verlag). This technique requires the choice of pairs of oligonucleotide primers surrounding the fragment which is to be amplified. Reference can be made, for example, to the technique described in the U.S. Pat. No. 4,683,202. The amplified fragments can be identified, for example after agarose or polyacrylamide gel electrophoresis, or after a chromatographic technique such as filtration on gel or ion-exchange chromatography, then sequenced. The specificity of the amplification can be controlled using as a primer the nucleotide sequences of polynucleotides of the invention as a matrix, plasmids containing these sequences or also the derived amplification products. The amplified nucleotide fragments can be used as reagents in hybridization reactions in order to show the presence, in a biological sample, of a target nucleic acid with a sequence which complements that of said amplified nucleotide fragments.

The invention is also aimed at the nucleic acids which are able to be obtained by amplification using primers according to the invention.

Other techniques for the amplification of target nucleic acid can be advantageously used as an alternative to PCR (PCR-like) using primer couples of nucleotide sequences according to the invention. By PCR-like is meant all of the methods using direct or indirect reproductions of the nucleic acid sequences, or in which the marking systems have been amplified, these techniques are of course known, in general these involve amplification of the DNA by a polymerase; when the original sample is an RNA it is advantageous to carry out a reverse transcription in advance. Currently very many processes exist which allow this amplification, such as for example the SDA (Strand Displacement Amplification) technique (Walker et al., 1992, Nucleic Acids Res. 20: 1691), the TAS technique (Transcription-based Amplification System) described by Kwoh et al. (1989, Proc. Natl. Acad. Sci. USA, 86, 1173), the 3SR (Self-Sustained Sequence Replication) technique described by Guatelli et al. (1990, Proc. Natl. Acad. Sci. USA 87: 1874), the NASBA (Nucleic Acid Sequence Based Amplification) technique described by Kievitis et al. (1991, J. Virol. Methods, 35.273), the TMA (Transcription Mediated Amplification) technique, the LCR (Ligase Chain Reaction) technique described by Landegren et al. (1988, Science 241.1077), the RCR (Repair Chain Reaction) technique described by Segev (1992, Kessler C. Springer Verlag, Berlin, New-York, 197-205), the CPR (Cycling Probe Reaction) technique described by Duck et al. (1990, Biotechnics, 9, 142), the Q-beta replicase amplification technique described by Miele et al. (1983, J. Mol. Biol., 171.281). Certain of these techniques have since been perfected.

In the case where the target polynucleotide to be detected is an mRNA, advantageously, before the implementation of an amplification reaction using the primers according to the invention or before the implementation of a detection process using the probes of the invention, a reverse transcriptase type enzyme in order to obtain a cDNA from the mRNA contained in the biological sample. The cDNA obtained will then serve as a target for the primers or the probes used in the amplification or detection process according to the invention.

The probe hybridization technique can be carried out in various ways (Matthews et al., 1988, Anal. Biochem., 169, 1-25). The most common method consists of immobilizing the nucleic acid extracted from the cells of different tissues or cells in culture on a support (such as nitrocellulose, nylon, polystyrene) and incubating, in well defined conditions, the target nucleic acid immobilized with the probe. After the hybridization, the probe excess is eliminated and the hybrid molecules formed are detected by an appropriate method (measurement of radioactivity, fluorescence or enzymatic activity linked with the probe).

According to another mode of application of the nucleic probes according to the invention, the latter can be used as capture probes. In this case, a probe, called a “capture probe”, is immobilized on a support and serves to capture by specific hybridization the target nucleic acid obtained from the biological sample to be tested and the target nucleic acid is then detected with a second probe, called a “detection probe”, marked with an easily detectable element.

Amongst the interesting fragments of nucleic acid, there must be mentioned in particular the anti-sense oligonucleotides, i.e. the structure of which allows, by hybridization with the target sequence, an inhibition of the expression of the corresponding product. The sense oligonucleotides which, by interaction with proteins involved in the regulation of the expression of the corresponding product, induce either an inhibition, or an activation of this expression must also be mentioned.

Preferably, the probes or primers according to the invention are immobilized on a support, covalently or non-covalently. In particular, the support can be a DNA chip or a high density filter, which are also subjects of the present invention.

By DNA chip or high density filter is meant a support on which DNA sequences are fixed, each of them being able to be located by its geographical location. These chips or filters differ mainly in their size, the support material, and optionally the number of DNA sequences which are fixed to it.

The probes or primers according to the present invention can be fixed on solid supports, in particular DNA chips, by different production processes. In particular, a synthesis in situ can be carried out by photochemical orientation or by ink-jet. Other techniques consist of carrying out a synthesis ex situ and fixing the probes onto the DNA chip support by mechanical, electronic or ink-jet orientation. These different processes are known to a person skilled in the art.

A nucleotide sequence (probe or primer) according to the invention thus allows the detection and/or the amplification of specific nucleic sequences. In particular, the detection of said sequences is facilitated when the probe is fixed on a DNA chip, or to a high density filter.

The use of DNA chips or high density filters in fact allows determination of the expression of genes in an organism having a gene sequence close to cyanophage S-2L.

The gene sequence of cyanophage S-2L, containing the identification of all of the genes of this organism, as presented in the present invention, serves as a basis for the construction of these DNA chips or filters.

The preparation of these filters or chips consists of synthesizing oligonucleotides, corresponding to the 5′ and 3′ ends of the genes. These oligonucleotides are chosen using the gene sequence and its annotations disclosed by the present invention. The pairing temperature of these oligonucleotides at the corresponding places on the DNA must be approximately the same for each oligonucleotide. This allows the preparation of DNA fragments corresponding to each gene by using appropriate PCR conditions in a highly automated environment. The amplified fragments are then immobilized on filters or supports made of glass, silicon or synthetic polymers and these media are used for the hybridization.

The availability of such filters and/or chips and of the corresponding annotated gene sequence allows the study of the expression of large groups, or even of all the genes in viruses close to cyanophage S-2L, by preparing the complementary DNAs, and by hybridizing them with the DNA or with the oligonucleotides immobilized on the filters or chips. Also, the filters and/or the chips allow the study of the variability of the strains by preparing the DNA of these viruses and by hybridizing them with the DNA or with the oligonucleotides immobilized on the filters or the chips.

The differences between the gene sequences of the different strains or species can greatly affect the intensity of the hybridization and, consequently, influence the interpretation of the results. It can therefore be necessary to have the exact sequence of the genes of the strain which is to be studied.

The use of high density filters and/or chips allows new knowledge to be obtained about the regulation of genes in organisms which are important in industry, and in particular recombinant bacteria incorporating genes of cyanophage S-2L propagated in diverse conditions. It also allows a rapid identification of the differences between the genomes of the strains used in various industrial applications.

Moreover, the DNA chips or the filters according to the invention, containing specific probes or primers of cyanophage S-2L, are very advantageous elements of kits or are necessary for the detection and/or the quantification of the expression of genes of cyanophage S-2L in recombinant bacteria integrating these genes.

In fact the control of gene expression is a critical point for the metabolic routes of cyanophage S-2L, either allowing the expression of one or more new genes, or modifying the expression of genes already present in the cell. The present invention provides the group of sequences naturally active in cyanophage S-2L allowing gene expression. It thus allows the determination of the group of sequences expressed in cyanophage S-2L. It also provides a tool which allows the locating of genes the expression of which follows a given pattern. For this purpose, the DNA of all or some of the genes of cyanophage S-2L can be amplified using primers according to the invention, then fixed to a support such as for example glass or nylon or a DNA chip, in order to create a tool which allows the expression profile of these genes to be studied. This tool, constituted by this support containing the coding sequences serves as a hybridization matrix for a mixture of marked molecules reflecting the messenger RNAs expressed in the cell (in particular the marked probes according to the invention). By repeating this experiment at different times and combining all of this data using appropriate processing, the expression profiles of all of these genes are then obtained. Knowledge of the sequences which follow a given regulation pattern can also be useful for researching in a targeted manner, for example by homology, other sequences which follow the same regulation pattern overall, but in a slightly different way. In addition, it is possible to isolate each control sequence present upstream of the segments which act as probes and to monitor their activity using appropriate means such as a reporter gene (luciferase, 6-galactosidase, GFP). These isolated sequences can then be modified and assembled by metabolic engineering with sequences which are of interest because of their optimal expression.

The present invention provides the list of genes coding or able to code for proteins regulating the transcription of the genes of cyanophage S-2L. Modifying the structure or the integrity of these genes can allow modification of the expression of the target genes controlled by target promoters of these regulators. The information given also allows a person skilled in the art to choose the appropriate regulator or regulators for the desired application as well as their target, which allows optimization of the expression of genes which are of interest. The use of the tools previously described as DNA chips, also allows all of the genes the regulation of which is modified by this inactivation to be located. It is thus possible to select a control sequence group corresponding, as closely as possible, to the same type of regulation. These sequences can then be used to control the expression of genes which are of interest.

According to another aspect, the invention relates to polypeptides comprising:
a) a polypeptide encoded by a nucleotide sequence according to the invention as defined previously, in particular a polypeptide encoded by an ORF;
b) a polypeptide having at least 80% preferably 85%, 90%, 95% and 98% identity with a polypeptide from a);
c) a biologically active fragment with at least 5, 7, 10 amino acids of a polypeptide according to a) or b);
d) a polypeptide modified with a polypeptide according to the invention, or as defined in a), b), or c).
   The invention preferably relates to:
a) the polypeptides of cyanophage S-2L of sequences SEQ ID No. 2 to SEQ ID No. 527, encoded respectively by ORFs 2 to 527,
b) the 54 polypeptides mentioned in Table 1 (SEQ ID No. 14, 18, 26, 68, 86, 92, 105, 109, 134, 142, 143, 148, 152, 169, 175, 187, 208, 211, 234, 246, 250, 257, 264, 286, 298, 316, 332, 342, 347, 348, 351, 355, 364, 365, 369, 370, 392, 395, 406, 418, 422, 425, 429, 432, 433, 454, 464, 466, 472, 484, 489, 494, 500.
c) the 14 polypeptides of cyanophage S-2L, shown in Table 1 as having a very significant homology, of sequence SEQ ID No. 86, 92, 152, 175, 234, 257, 298, 316, 395, 406, 425, 484.
   The invention also relates to:
d) the polypeptides having at least 80% preferably 85%, 90%, 95% and 98% identity with a polypeptide from a), b), c)
e) the biologically active fragments of the polypeptides from a), b), c), d)
f) the modified polypeptides from a), b), c), d), e).

Of course the invention relates in particular to the polypeptides involved in the biosynthesis of the D-bases and metabolic intermediates of this biosynthesis, in particular the peptide of sequence SEQ ID No. 175 with succinylate synthetase activity.

The phages the modifications of which are prereplicative, which is probably the case for cyanophage S-2Ls, have the coding sequences of proteins which are required for the biosynthesis of the modified bases, in the present case of the D-bases. Moreover, in as far as the D-bases are a part of the genome of cyanophage, the polymerase enzymes in particular DNA polymerase must be capable of having the D-base specifically as substrate instead of the A-base. The DNA polymerase of cyanophage S-2L is thus capable of distinguishing dDTP from dATP. Similarly, the transcription depends on a specific RNA polymerase and/or a specific sigma factor. Thus the invention relates, according to a preferred embodiment, to the specific polypeptides with DNA polymerase, RNA polymerase activity and related factors, in particular the peptides of sequence SEQ ID No. 92 and SEQ ID No. 234 which have specific activities of transcription of DNA comprising D-bases.

In the present description, the terms polypeptides, polypeptide sequences, peptides and proteins are interchangeable.

It must be understood that the invention does not relate to polypeptides in natural form, that is to say that they are not in their natural environment but that they have been able to be isolated or obtained by purification from natural sources, or obtained by genetic recombination, or by chemical synthesis, and that they can then comprise unnatural amino acids such as will be described subsequently.

By polypeptide having a certain identity percentage with another, which is also called an homologous polypeptide, is meant the polypeptides having certain modifications in relation to the natural polypeptides, in particular a deletion, addition or substitution of at least one amino acid, a truncation, an extension, a chimeric solution and/or a mutation, or the polypeptides having post-translational modifications. Among the homologous polypeptides, those the amino acid sequence of which has at least 80%, preferably 85%, 90%, 95% and 98% homology with the amino acid sequences of the polypeptides according to the invention are preferred. In the case of a substitution, one or more consecutive or non-consecutive amino acid(s) are replaced with “equivalent” amino acids. The expression “equivalent amino acids” here is meant to designate any amino acid which is capable of being substituted for one of the amino acids of the base structure without however essentially modifying the biological activities of the corresponding peptides as defined subsequently.

These equivalent amino acids can be determined either on the basis of their homology of structure with the amino acids for which they are substituted, or on results of biological activity comparison tests of which can be carried out between the different polypeptides.

By way of an example, the substitution possibilities which can be carried out without resulting in a great modification of the biological activity of the corresponding modified polypeptide are mentioned. Thus leucine can be replaced by valine or isoleucine, aspartic acid by glutamine acid, glutamine by asparagine, arginine by lysine, etc. the reverse substitutions being naturally envisageable under the same conditions.

The homologous polypeptides also correspond to the polypeptides encoded by the homologous or identical nucleotide sequences, as defined previously and thus include in the present definition polypeptides which are mutated or which correspond to variations between or within species, being able to exist in cyanophage S-2L, and which correspond in particular to truncations, substitutions, deletions and/or additions, of at least one amino acid residue.

It is understood that the identity percentage between two polypeptides is calculated in the same way as between two nucleic acid sequences. Thus, the identity percentage between two polypeptides is calculated after optimal alignment of these two sequences, on a maximum homology window. In order to define said maximum homology window, the same algorithms can be used as for the nucleic acid sequences.

By biologically active fragment of a polypeptide according to the invention, is meant in particular a polypeptide fragment comprising at least 5 amino acids, preferably at least 7, 10, 15, 25, 50, 75, 100, 150, 200, 250, 300 amino acids, having at least one of the biological characteristics of the polypeptides according to the invention, in particular in that it is generally capable of carrying out even a partial activity, such as for example:
an (metabolic) enzyme activity or an activity which can be involved in the biosynthesis or biodegradation of organic or inorganic compounds;
a structural activity (cell envelope etc.);
an activity in the process of replication, amplification, preparation, transcription, translation or processing, in particular of DNA, RNA or proteins
and quite particularly an activity involved in the biosynthesis of D-bases.

The polypeptide fragments can correspond to isolated or purified fragments naturally present in the strains of cyanophage S-2L, or to fragments which can be obtained by cleavage of said polypeptide by a proteolytic enzyme such as trypsin or chymotrypsin or collagenase, by a chemical reagent (cyanogen bromide, CNBr) or by placing said polypeptide in a very acidic environment (for example at pH=2.5). Polypeptide fragments can also be prepared by chemical synthesis, from hosts transformed by an expression vector according to the invention which contain a nucleic acid allowing the expression of said fragment, and placed under the control of the appropriate regulation and/or expression elements.

By “modified polypeptide” of a polypeptide according to the invention, is meant a polypeptide obtained by genetic recombination or by chemical synthesis such as described subsequently, which has at least one modification in relation to the normal sequence. These modifications can in particular be carried on amino acids necessary for the specificity or efficiency of the activity, or at the origin of the structural conformation, the charge, or the hydrophobicity of the polypeptide according to the invention. Thus polypeptides with equivalent, increased or reduced activity, or with equivalent, narrower or wider specificity can be created. Amongst the modified polypeptides, the polypeptides in which up to five amino acids can be modified, truncated at the N or C terminal end, or deleted, or added should be mentioned.

As is shown, the modifications of a polypeptide are aimed in particular at:
allowing its use in biosynthesis or biodegradation processes of organic or inorganic compounds,
allowing its use in replication, amplification, repair and transcription regulation, translation, or maturation processes in particular of DNA, RNA, or proteins,
allowing its improved secretion,
modifying its solubility, the efficiency or specificity of its activity, or also facilitating its purification.

The chemical synthesis also has the advantage of being able to use unnatural amino acids or non-peptide bonds. Thus, it can be advantageous to use unnatural amino acids, for example in D form, or amino acid analogues, in particular sulphurized forms.

In another feature, preferably, the subject of the invention is a polypeptide according to the invention, characterized in that it is a polypeptide of cyanophage S-2L or one of its representative fragments involved in the metabolism of nucleotides, purines, pyrimidines or nucleosides.

In another feature, preferably, a subject of the invention is a polypeptide according to the invention, characterized in that it is a polypeptide of cyanophage S-2L or one of its representative fragments involved in the replication process , and in that it is chosen from the polypeptides of sequence SEQ ID No. 14, 18, 142, 355, 429, 454 and one of their fragments.

The invention very advantageously relates to polypeptides of cyanophage S-2L with at least 7 amino acids and having an adenylosuccinate synthetase activity. Preferably, such fragments include the GSTGKG unit. Moreover biological results (specific metabolism of cyanophage S-2L capable of synthesizing and polymerizing DNA incorporating D-bases), the inventors in fact identified consensus sites in particular the zones which are the phosphate and IMP binding sites. In particular the fragment QYGSTGKG is found, which is close to the Prosite signature QWGDEGKG attributed to adenylosuccinate synthetase, or the fragment GSTGKG close to the fragment GDEGKG which is common to Escherichia coli, Methanobacterium thermoautotrophicum, Pyrococcus horikoshii OT3. The inventors identified significant homologies for adenylosuccinate synthetase, helicase, sigma factor activities, these three activities being a priori closely and directly linked with the specific metabolism of the D-bases.

In another feature, preferably, a subject of the invention is a polypeptide according to the invention, characterized in that it is a polypeptide of cyanophage S-2L or one of its fragments involved in the transcription process, and in that it is chosen from the polypeptides of sequence SEQ ID No. 92, 143, 187 and one of their representative fragments.

In another aspect, preferably, a subject of the invention is a polypeptide according to the invention, characterized in that it is an envelope polypeptide of cyanophage S-2L or one of its fragments, and in that it is chosen from the polypeptides corresponding to ORFs 169, 316, 351, 392, 395, 406, 422, 425 and one of their representative fragments.

In another feature, preferably, a subject of the invention is a polypeptide according to the invention, characterized in that it is a polypeptide of cyanophage S-2L or one of its representative fragments involved in the rerouting of the cell machinery or in the intermediate metabolism.

In another feature, preferably, a subject of the invention is a polypeptide according to the invention, characterized in that it is a polypeptide of cyanophage S-2L or one of its representative fragments involved in the virulence process, in particular the polypeptide of sequence SEQ ID No. 247 and one of its representative fragments.

In another aspect, preferably, a subject of the invention is a polypeptide according to the invention, characterized in that it is a polypeptide of cyanophage S-2L or one of its fragments involved in the functions relating to transposons, in particular the polypeptide of sequence SEQ ID No. 208 and one of its representative fragments.

However it must be noted that a living organism is a whole and must be treated as such. Thus, in order to be able to develop and exhibit its properties, any organism requires interactions between the different metabolic routes. Thus, the classification given above must not be considered as being limitative, a gene being able to be involved in several distinct metabolic routes.

A subject of the present invention is also the nucleotide and/or polypeptide sequences according to the invention, characterized in that said sequences are recorded on a recording medium, the form and nature of which facilitate the reading, analysis and/or exploitation of said sequence or sequences. These media can also contain other information extracted from the present invention, in particular analogies with the sequences which are already known, as mentioned in Table 1 and/or information relating to the nucleotide and/or polypeptide sequences of other microorganisms in order to facilitate the comparative analysis and exploitation of the results obtained.

Amongst said recording media, the media which are readable by a computer, such as the magnetic, optical, electrical or hybrid media, in particular floppy disks, CD-ROMs, servers are preferred. Such recording media are also a subject of the invention.

The recording media according to the invention, with the information provided, are very useful for choosing nucleotide primers or probes for determining the genes in cyanophage S-2L or strains close to this organism. Similarly, the use of these media for the study of genetic polymorphism of a strain close to cyanophage S-2L, in particular by determining the colinearity regions, is very useful in that these media provide not only the nucleotide sequence of the genome of cyanophage S-2L, but also the genome organization in said sequence. Thus, the uses of recording media according to the invention are also subjects of the invention.

A process for studying the genetic polymorphism between the strains close to cyanophage S-2L, by determining the colinearity regions, can comprise the stages of
fragmentation of the chromosomal DNA of said other strain (sonication, digestion),
sequencing of the DNA fragments,
analysis of homology with the genome of cyanophage S-2L (SEQ ID No. 1).

This process which comprises a stage of analysis of homology with the genome of cyanophage S-2L, in particular using a recording medium, is also a subject of the invention.

The analysis of homology between different sequences is advantageously carried out using sequence comparison software, such as Blast software, or the GCG software package, described previously.

The invention is also aimed at a nucleotide sequence such as described previously, immobilized on a support, covalently or non-covalently, in particular a high density filter or a DNA chip.

The invention is also aimed at a nucleotide sequence such as described previously for the detection and/or amplification of nucleic sequences.

According to one embodiment such a detection and amplification process comprises for example the following stages:
a) optionally, isolation of the DNA from the biological sample to be analyzed, or obtaining of an cDNA from the RNA of the biological sample;
b) specific amplification of the DNA of cyanophages S-2L using at least one primer according to the invention;
c) revealing the amplification products.

This process is based on the specific amplification of DNA, in particular by a chain reaction amplification.

A process comprising the following stages is also preferred:
a) bringing a nucleotide probe according to the invention into contact with a biological sample, the nucleic acid contained in the biological sample having, if appropriate, previously been made accessible for hybridization, under conditions which allow the hybridization of the probe with the nucleic acid of cyanophage S-2L;
b) revealing the hybrid optionally formed between the nucleotide probe and the DNA of the biological sample.

Such a process should not be limited to the detection of the presence of DNA contained in the verified biological sample, it can also used to detect the RNA contained in said sample. This process includes in particular Southern and Northern blots.

Thus, the present invention also includes a kit or set for the detection and/or identification of cyanophage S-2L, characterized in that it comprises the following elements:
a) a nucleotide probe according to the invention;
b) optionally, the reagents necessary for implementing a hybridization reaction;
c) optionally, at least one primer according to the invention as well as the reagents required for a DNA amplification reaction.

Similarly, the present invention also includes the kits or sets for the detection and/or identification of cyanophage S-2L, comprising the following elements:
a) a nucleotide probe, called a capture probe, according to the invention;
b) an oligonucleotide probe, called a revelation probe, according to the invention;
c) optionally, at least one primer according to the invention as well as the reagents required for a DNA amplification reaction.

The invention is also aimed at the cloning and/or expression vectors, which contain a nucleotide sequence according to the invention. The nucleotide sequences coding for polypeptides involved in the metabolism of nucleotides, purines, pyrimidines or nucleosides are in particular preferred.

The vectors according to the invention preferably comprise elements which allow the expression and/or secretion of nucleotide sequences in a determined host cell.

The vector must then comprise a promoter, translation initiation and termination signals, as well as appropriate regions of transcription regulation. It must be able to be maintained in a stable manner in the host cell and can optionally have particular signals which specify the secretion of the translated protein. These different elements are chosen and optimized by a person skilled in the art according to the host cell used. For this purpose, the nucleotide sequences according to the invention can be inserted into vectors with autonomous replication inside the chosen host, or be vectors which integrate into the chosen host.

Such vectors are prepared by methods commonly used by a person skilled in the art, and the resulting clones can be introduced into an appropriate host by means of standard methods, such as lipofection, electroporation, thermal shock, or chemical methods.

The vectors according to the invention are for example vectors of plasmid or viral origin. They are useful for transforming host cells in order to clone or express the nucleotide sequences according to the invention. The cyanophage S-2L itself can be used directly as vector.

The invention also comprises the host cells transformed by a vector according to the invention.

The host cell can be chosen from prokaryotic or eukaryotic systems, for example bacteria cells but also yeast cells or animal cells, in particular mammal cells. Insect cells or plant cells can also be used. The host cells preferred according to the invention are in particular prokaryotic cells. The cells which are transformed according to the invention can be used in recombinant polypeptide preparation processes according to the invention.

The processes for preparing a polypeptide which is of interest according to the invention in recombinant form, outside the natural environment, characterized in that they use a vector and/or a cell transformed by a vector according to the invention are themselves included in the present invention. The use of cyanophage S-2L for the production of such peptides is thus also a part of the invention.

Preferably, a cell transformed by a vector according to the invention is cultured under conditions which allow the expression of said polypeptide of interest and said recombinant peptide is recovered. The host cells according to the invention can also be used for the preparation of dietary compositions, which are themselves a subject of the present invention.

Such a process for obtaining proteins of interest of cyanophage S-2L comprises according to one embodiment the insertion of genes of interest of the genome of S-2L phage, typically by ligation, into cloning and expression vectors, under conditions which allow their expression by the replication machinery taking charge of a host organism such as E. coli, and the extraction of the proteins produced.

The hereditary messages of the phage recopied in the form of canonical DNA are able to express themselves as cyanobacteria genes. The messenger RNAs emitted after rewriting of the DNA of S-2L in E. coli are translated into proteins identical to those produced when infecting Synechococcus with S-2L.

According to a preferred embodiment the polypeptides of interest are proteins involved in the metabolism of D-bases, in particular succinyladenylate synthetase.

It was stated above that a D-base is probably formed by pre-replicative modification and that cellular genes were recruited for this purpose, two biosynthesis routes presenting themselves to form dDTP from a canonic deoxynucleotide, dAMP or dGMP.

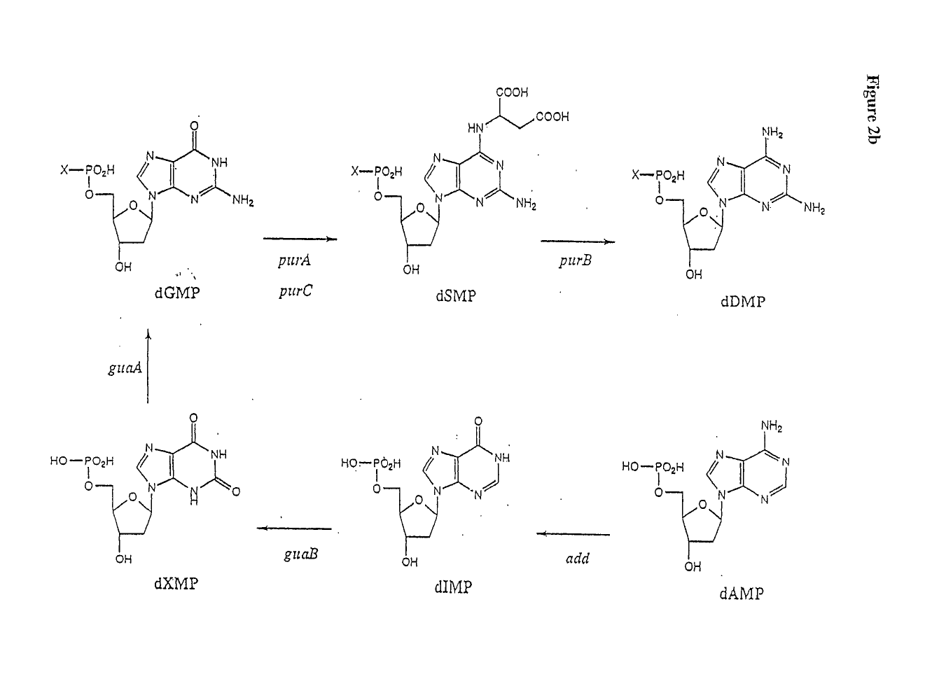

According to the first route (FIG. 2a), the activated monomer dATP is firstly hydrolyzed to dAMP by an enzyme of the type coded by DUT in E. coli (9) or from the product of the mutT gene (9), which has the twofold effect of blocking access of dATP to DNA synthesis and providing the precursor of DMP. The biosynthesis of the latter is carried out following the two successive reactions converting IMP into GMP in the cell metabolism (9); the nucleotide is finally activated in DDTP in two phosphorylation stages.

According to the second route (FIG. 2b), dDMP is obtained by applying to dGMP the two reactions converting IMP into AMP in the cells (9). If it also takes dATP as precursor, this second route is longer since dGMP must previously be synthesized via dIMP. All along this second route, three specific and mutagenic dNTPs are formed (dIMP, dXMP and dSMP), compared with just one (diGMP) in the first (FIG. 2a).

As is described subsequently the inventors have succeeded in identifying an ORF coding for an enzyme of the second route, succinyladenylate synthetase.

According to another preferred embodiment, the polypeptides of interest are polymerases of cyanophages S-2L, capable of polymerizing D-bases, which allows the propagation of the nucleic acids incorporating D-bases in vitro and in vivo.

The inventors obtain in particular DNA polymerases which are peculiar to the duplex with high stability and unable to replicate dA taken as a constituent of the matrix or as triphosphate monomer. These DNA polymerases are typically obtained by a process comprising a stage of expression, outside the natural environment, of the gene of said DNA polymerase in recombinant bacteria.

According to a preferred embodiment the polypeptides of interest are polypeptides which are capable of modifying the transcription of the DNA of host cells of cyanophage S-2L.

In fact the custom-made transcription of the genome of S-2L of an RNA polymerase, even if it is not coded in the phage. It is known that T4 enzymes alter the RNA polymerase of E. coli. The promoters present in the genome of S-2L deviate from the consensus known to a person skilled in the art (TATA box in particular). It is probable that transcription initiation factors (sigma etc.) will be coded or modified by the phage, or even that they will be taken into the capsid to allow the start of the viral program. Whatever the case, the sequencing carried out by the inventors allows identification without excessive effort of certain genes of S-2L which are responsible for the control of the transcription by chemical alteration of DNA.

As has been stated, the host cell can be chosen from prokaryotic or eukaryotic systems. In particular, it is possible to identify nucleotide sequences according to the invention, which facilitate secretion in such a prokaryotic or eukaryotic system. A vector according to the invention carrying such a sequence can thus be advantageously used for the production of recombinant proteins, which are designed to be secreted. The purification of these recombinant proteins of interest is facilitated by the fact that they are present in the supernatant of the cellular culture rather than inside host cells.

The polypeptides according to the invention can also be prepared by chemical synthesis. Such a preparation process is also a subject of the invention. A person skilled in the art knows the chemical synthesis processes, for example the techniques using solid phases (see in particular Steward et al., 1984, Solid phase peptides synthesis, Pierce Chem. Company, Rockford, 111, 2nd ed., (1984)) or techniques using partial solid phases, by fragment condensation or by a synthesis in standard solution. The polypeptides obtained by chemical synthesis and which are able to comprise corresponding unnatural amino acids are also included in the invention.

The invention also includes the hybrid polypeptides which include at least the sequence of one polypeptide according to the invention, and the sequence of a polypeptide which is able to induce an immune response in a human or animal. The invention also comprises the nucleotide sequences which code for such hybrid polypeptides, or the vectors which contain these nucleotide sequences. This coupling between a polypeptide according to the invention and an immunogenic polypeptide of interest, can be carried out by chemical route, or by biological route. Thus, according to the invention, it is possible to introduce one or more bonding element(s), in particular amino acids, in order to facilitate the coupling reactions between the polypeptide according to the invention, and the immunostimulation polypeptide, the covalent coupling of the immunostimulation antigen being able to be carried out at the N or C-terminal end of the polypeptide according to the invention. The bifunctional reagents which allow this coupling are determined according to the end chosen for carrying out this coupling, and the coupling techniques are well known to a person skilled in the art.

The conjugates produced by a coupling of peptides can also be prepared by genetic recombination. The hybrid peptide (conjugated) can in fact be produced by recombinant DNA techniques, by insertion or addition to the DNA sequence coding for the polypeptide according to the invention, of a sequence coding for the antigen, immunogen or hapten peptide or peptides. These techniques for the preparation of hybrid peptides by genetic recombination are well known to a person skilled in the art (see for example Makrides, 1996, Microbiological Reviews 60.512-538).

Preferably, said immunitary polypeptide is chosen from the group of peptides containing the toxoids, in particular diphteria toxoid or tetanus toxoid, the proteins derived from Streptococcus (such as the protein bonding with human blood albumin), the membrane OmpA proteins and the outer membrane protein complexes, the vesicles of outer membranes or heat-shock proteins.

The nucleotide and vector sequences, coding for a hybrid polypeptide according to the invention are also a subject of the invention.

The hybrid polypeptides according to the invention are very useful for obtaining monoclonal or polyclonal antibodies, which are capable of specifically recognizing the polypeptides according to the invention. In fact a hybrid polypeptide according to the invention allows potentiation of the immune response, against the polypeptide according to the invention coupled with the immunogenic molecule. Such monoclonal or polyclonal antibodies, their fragments, or the chimeric antibodies, recognizing the polypeptides according to the invention, are also subjects of the invention.

The specific monoclonal antibodies can be obtained according to the standard method of hybridoma culture described by Köhler and Milstein (1975, Nature 256, 495).

The antibodies according to the invention are for example chimeric antibodies, humanized antibodies, Fab, or F(ab′)² fragments. They can also be presented in the form of an immunoconjugate or marked antibodies in order to obtain a detectable and/or quantifiable signal.

The antibodies according to the present invention can in particular be used in order to detect an expression of a gene of cyanophage S-2L. In fact the presence of the expression product of a gene recognized by a specific antibody of said expression product can be detected by the presence of an antigen-antibody complex formed after bringing into contact a recombinant bacterium expressing a given gene of interest of cyanophage S-2L and an antibody according to the invention. The bacterial strain used can have been “prepared”, i.e. centrifuged, lysed, placed in an appropriate reagent for the constitution of the medium which is conducive to the immunological reaction. In particular, a process for the detection of the expression of a gene, corresponding to a Western blot, which can be carried out after polyacrylamide gel electrophoresis of a lysate of the bacterial strain, in the presence or in the absence of reducing conditions (SDS-PAGE). After migration and separation of the proteins on the polyacrylamide gel, said proteins are transferred onto an appropriate membrane (for example nylon) and the presence of the protein or the polypeptide of interest is detected, by bringing into contact said membrane and an antibody according to the invention.

The polypeptides and the antibodies according to the invention can advantageously be immobilized on a support, in particular a protein chip. Such a protein chip is a subject of the invention, and can also contain at least one polypeptide of a microorganism other than cyanophage S-2L or an antibody directed against a compound of a microorganism other than cyanophage S-2L. The protein chips or high density filters containing proteins according to the invention can be created in the same way as the DNA chips according to the invention. In practice, the synthesis of the polypeptides fixed directly onto the protein chip can be carried out, or a synthesis can be carried out ex situ followed by a stage of fixation of the synthesized polypeptide onto said chip. The latter method is preferable, when large proteins, which are advantageously prepared by genetic engineering, are to be fixed onto the support. However, if only peptides are to be fixed onto the support of said chip, it can be more advantageous to proceed to synthesizing said peptides directly in situ.

Preferably, an antibody according to the invention is fixed onto the support of the protein chip, and the presence of the corresponding antigen, specific to cyanophage S-2L or a related microorganism is detected.

A protein chip described above can be used for the detection of gene products, in order to establish an expression profile of said genes, complementing a DNA chip according to the invention.

The protein chips according to the invention are also extremely useful for proteomics testing, which studies the interactions between the different proteins of a given microorganism. In a simplified manner, representative peptides of the different proteins of an organism are fixed onto a support. Then said support is brought into contact with marked proteins, and after an optional stage of rinsing, interactions between said marked proteins and the peptides fixed on the protein chip are detected.

Thus, the protein chips comprising a polypeptide sequence according to the invention or an antibody according to the invention are a subject of the invention, as well as the kits or sets containing them.

Preferably, the primers and/or probes and/or polypeptides and/or antibodies according to the present invention used in processes according to the present invention are chosen from the specific primers and/or probes and/or polypeptides and/or antibodies of cyanophage S-2L.

A subject of the present invention is also the strains of cyanophage S-2L and/or of related microorganisms containing one or more mutation(s) in a nucleotide sequence according to the invention, in particular an ORF sequence, or their regulating elements (in particular promoters).

According to the present invention, the strains of cyanophage S-2L having one or more mutation(s) in the nucleotide sequences coding for polypeptides involved in the metabolism of the D-bases, replication and transcription are preferred.

Said mutations can lead to an inactivation of the gene, or in particular when they are situated in the regulating elements of said gene, to its overexpression.

Thus, strains of cyanophage S-2L which overexpress a polypeptide according to the invention are sought in particular, involved in the functions relating to the synthesis of D-bases or of polynucleotides incorporating at least one D-base.

The prior art displays knowledge of the specific metabolism of cyanophage S-2L, leading to the synthesis of D-bases instead of A-bases. However, until now, without knowing the exact sequence of cyanophage S-2L, a person skilled in the art did not have at his disposal the ORF coding sequences and therefore could not in particular efficiently clone a given ORF sequence, test the corresponding biological activity and express polypeptides of interest. This type of process is now possible thanks to the sequencing of the genome of cyanophage S-2L which was carried out by the inventors.

Even without knowing precisely at this stage the synthesis route of the D-bases, the inventors have succeeded in identifying coding sequences involved in this metabolic route. By successive testing, but without excessive effort for a person skilled in the art, of the biological function of the ORF capable of intervening in this metabolic route from the results obtained (more specifically the ORFs of the polypeptides group intervening in the metabolism of nucleotides, purines, pyrimidines or nucleosides), the inventors can thus locate those which code for the proteins determining this route.

According to another feature the invention also relates to the use of the polypeptide sequences as described previously for the production of D-bases and/or polynucleotide sequences comprising D-bases. These polynucleotide sequences are in particular DNA or RNA sequences, in particular mRNA.

According to another feature the invention relates to a process for obtaining D-bases and/or polynucleotides of interest comprising at least one D-base, said process comprising the culture of a microorganism containing at least one nucleotide sequence of cyanophage S-2L coding for at least one polypeptide involved in the synthesis of D-bases, under appropriate conditions for the development of the vector and the synthesis of D-bases. Typically the microorganism cultured comprises a vector as described previously containing said nucleotide coding sequence or sequences of cyanophage S-2L.

According to one embodiment such a process comprises:
the addition to a medium comprising the substrates required for obtaining D-bases, of an extract or mixture of extracts of recombinant bacteria expressing at least one gene of cyanophage S-2L involved in the synthesis of D-bases
if appropriate the extraction of D-bases and/or said polynucleotides of interest.

According to one embodiment such a process comprises:
the preparation of at least one DNA sequence coding for a polypeptide capable of provoking the synthesis of at least one D-base in a host microorganism
the cloning of said coding sequence in a vector which is capable of being transferred into and replicating in said host microorganism, this vector comprising the elements necessary for the expression of said coding sequence
the transfer of the vector comprising said coding sequence into a microorganism capable of producing the enzymes of the D-base synthesis directed by said coding sequence
the culture of the microorganism under appropriate conditions for the development of the vector and the synthesis of the D-bases
if appropriate the extraction of D-bases and/or of said polynucleotides of interest.

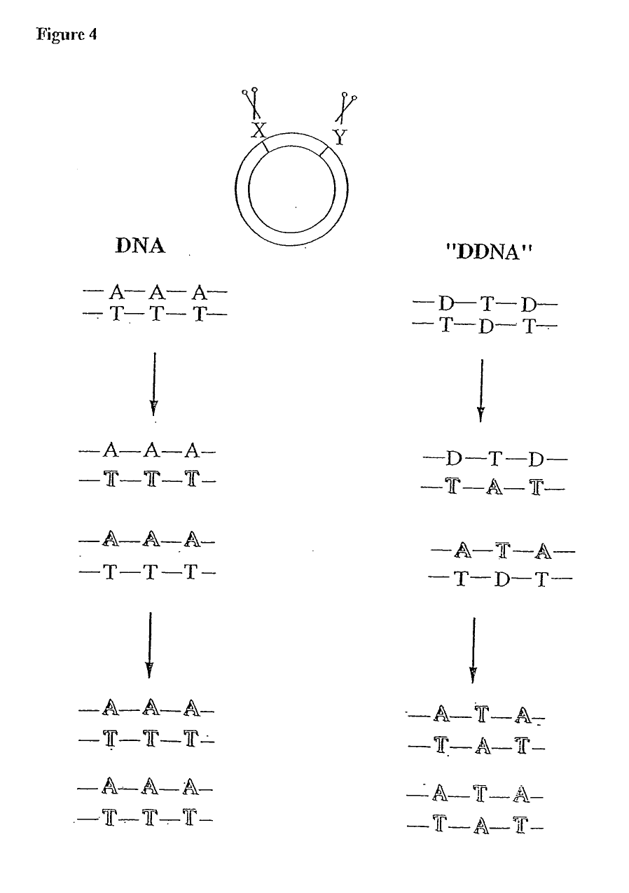

As is described subsequently, the inventors have succeeded in cloning DNA containing D-bases, using restriction enzymes the restriction sites of which do not have an A-base, in particular SmaI (site CCCGGG), SacII (site CCGCGG), MspI (site CCGG), BspRI (site GGCC). The inventors have shown that restriction enzymes comprising at least one A-base do not hydrolyze the DNA of S-2L: BamHI (GCATCC), EcoRI (GAATTC), HindIII (AAGCTT), Sau3AI (GATC). For this purpose the inventors challenged a technical assumption, namely that the cloning of a DNA comprising D-bases could lead to ambiguities in copying during cloning. In fact, as shown in FIG. 4, the cloning of “D DNA” in E. Coli, is capable of leading to sequences which are different from those produced by the cloning of “A DNA”.

According to another feature the invention relates to a process for obtaining D-bases and/or polynucleotides of interest comprising at least one D-base, said process comprising:
the addition, to a medium comprising the substrates required for obtaining D-bases, of the expression product of at least one gene of cyanophage S-2L involved in the synthesis of D-bases, in order to produce D-bases and/or polynucleotides of interest comprising at least one D-base
if appropriate the extraction of the D-bases and/or said polynucleotides of interest.

In the processes mentioned above, by synthesis of D-bases and/or polynucleotides comprising at least one D-base, is meant that the conditions of the synthesis are such that only or essentially D-bases, or only or essentially polynucleotides comprising at least one D-base, or at the same time D-bases and polynucleotides comprising at least one D-base are obtained in desired quantities. The quantities of D-bases and polynucleotides comprising at least one D-base produced depend in particular on the control of the expression of proteins involved in the syntheses of the D-bases and in the incorporation of the D-bases during the extension of the polynucleotide chains.

According to another feature the invention relates to a process for obtaining polynucleotides of interest comprising at least one D-base, said process comprising the culture of a microorganism containing at least one nucleotide sequence of cyanophage S-2L coding for at least one polypeptide involved in the extension of said polynucleotides with incorporation of D-bases, DNA polymerase in particular, in appropriate conditions for the development of the vector and the extension of said polynucleotides.

According to another feature the invention relates to the use of cyanophage S-2L for the production of reagents which are useful for PCR or PCR-like reactions involving D-bases. In particular according to a preferred embodiment these reagents are dDTP monomers.

The dDTP monomer is a good substrate of the DNA polymerases of cyanophage S-2L, and matrices comprising the D-base are efficiently replicated (1). The biotechnological production of dD, dDMP and dDTP thus applies to the PCR techniques, increasing the thermal stability of the duplexes, or masking and unmasking many restriction sites (10). It is understood that this production is not a production in the natural environment, production in the natural environment meaning production by the cyanophage S-2L itself.

The invention also relates to a process for the production of polynucleotides of interest comprising at least one D-base, said process comprising a stage of amplification, in the presence of cyanophage D polymerase and appropriate primers, of polynucleotides comprising at least one D-base.

Using this process, according to a technique of PCR or PCR-like type, from a polynucleotide of interest comprising at least one D-base with a known sequence, a large number of copies of this nucleotide are obtained.

According to one embodiment the gene involved in the synthesis of polynucleotides of interest comprising at least one D-base is the gene of succinyladenylate synthetase. In fact, succinyladenylate synthetase (ddba) catalyzes the reaction of dGMP to dSMP which is itself transformed into dDMP (FIG. 2).

According to one embodiment the polynucleotides of interest are nucleosides of therapeutic interest.

According to one embodiment, the polynucleotides of interest are produced by hemisynthesis or by fermentation.

The invention also relates to a process for the selection of compounds capable of stimulating or inhibiting the synthesis of D-bases and/or polynucleotides of interest incorporating at least one D-base, comprising the addition to the synthesis medium of the tested compound and comparison of the synthesis in the presence and in the absence of said compound.

According to another feature the invention relates to the use of the nucleotide sequences of cyanophage S-2L such as described previously in order to test their function in the metabolism of nucleotides, purines, pyrimidines or nucleosides, replication and transcription.

According to another feature the invention relates to the use of cyanophage S-2L for the determination of genes which allow the repair of the mismatches G:T or iG:T which occur by deamination.

The D-base itself is known to be a mutagen in E. coli. This could be explained by the fact that the deamination of D at position 2 leads to isoguanine (iG), for which it has recently been shown that the deoxynucleoside is mutagenic (M. Bouzon, P. Marliere, results not published). The deamination of D at position 6 leads to guanine. The fact that this last deamination reaction occurs after incorporation of D into the DNA, will result in a mutation in the following replication cycle. Thanks to the sequencing which has been carried out, the identification of genes which are able to repair the mismatches G:T or iG:T which occur by deamination is now possible.

According to another feature the invention relates to the use of cyanophage S-2L for the identification of genes and the production of proteins which are able to regenerate 5′-termini.

In fact the replication of the DNA of the cyanophage, the stability of which is high (7,8), could moreover require custom-made auxiliary proteins (helicase, SSB). The genome is constituted by a linear duplex, which supposes a regeneration machinery of the 5′-termini, such as the endonuclease used to resolve the concatemers in T7 (4), or the adduction protein in 5′ in phi29 (6), the activity of which could require the presence of D in their substrates.

According to another feature the invention relates to the use of cyanophage S-2L for the identification of genes capable of modulating the activity of the ribosomes.

In fact cyanophage S-2L is also able to form a ribonucleotide precursor which carries the D-base, in order to then reduce it to a corresponding deoxyribonucleotide, as occurs for the four bases of RNA (9). In this case, the transcription and translation of the phage genes could be carried out by using codons, or tRNAs as in T4 and T5, comprising this base. If such an option was taken by the phage, it is possible that certain of its genes modulate the activity of the ribosomes.

According to another aspect the invention relates to the use of cyanophage S-2L for the identification or the production of compounds inhibiting the biosynthesis of puric nucleotides.

In fact the phage genomes specify a whole range of inhibitors which have as target cellular enzymes such as thymidylate synthetase, dUTPase, etc. (11). In the case of S-2L, the inventors can now identify inhibitors capable of affecting the biosynthesis of puric nucleotides.

The invention thus also relates to a process using such inhibitors to control the metabolism or the gene expression of cells capable of being infected by an cyanophage S-2L, in particular cyanobacteria.

To the extent that the control of the metabolism of the nucleic acids or nucleosides, in particular of the DNA pyrimidines, is very useful in chemotherapy and gene therapy (2), the invention also relates to a process using such inhibitors in order to control this metabolism.

Other aspects and advantages of the invention will become apparent when reading the following description illustrated by the figures in which:

FIG. 1 represents a few examples of modified bases

FIGS. 2a and 2b represent two possible biosynthesis routes for the synthesis of D-bases by cyanophage S-2L, the route of FIG. 2b being the most likely

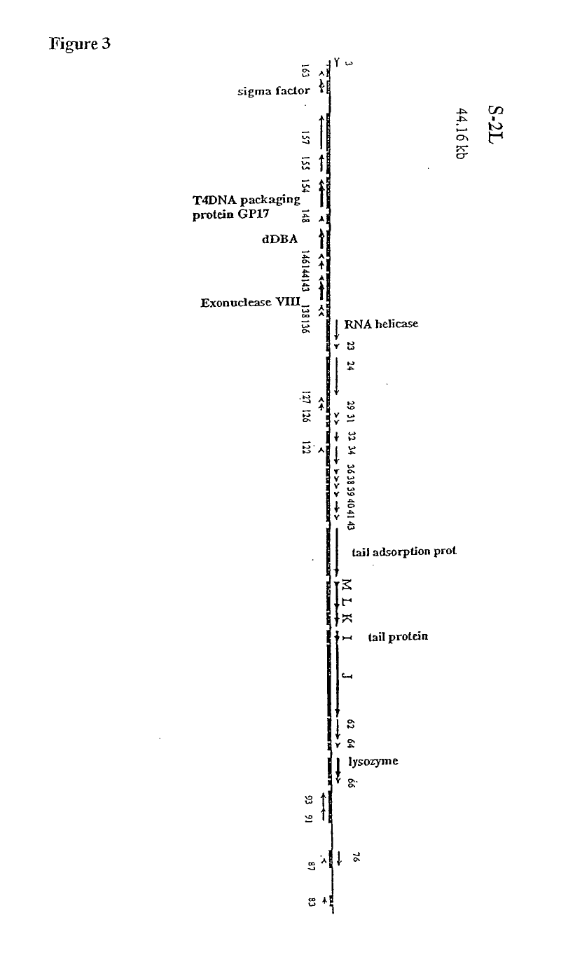

FIG. 3 schematically illustrates the genome of cyanophage S-2L

FIG. 4 schematically represents the potential difficulty of cloning genes incorporating D-bases in E. Coli.

Cyanophages S-2L are cultured in mass from the species Synechococcus elongatus (8). The DNA extracted is fragmented by sonication in order to constitute a shotgun bank cloned in a vector in E. coli. The clones are sequenced intensively on a sequencer until the genome is completely covered.

To the extent that ORFs are elucidated as homologous to known genes, they are expressed in E. coli or in Synechococcus, according to their supposed functions, in particular with the object of validating the functional hypotheses or exploring synthetic potentialities.

To this end, the supposed synthesis route intermediaries (FIG. 2) were synthesized according to the common methods of nucleoside and nucleotide chemistry. They are systematically subjected to extracts or mixtures of extracts of recombinant strains each expressing a gene of S-2L, in order to identify the enzymatic activities specified by the phage.

More precisely, the DNA of the S-2L phage was prepared from the Synechococcus elongatus culture lysate by adapting the techniques used in order to prepare the DNA of the λ-phage. This DNA was digested by different restriction enzymes, including SmaI, which made it possible to verify that the restriction profile obtained was identical to that described. Then, it was shown that the DNA of S-2L could be replicated in E. coli and sequenced according to the standard protocols, which led to the construction of a whole bank. This bank was constructed by insertion of DNA fragments digested by the enzyme CviJI (with a size comprised between 3 and 5 kb) in the plasmid pBAM digested by the enzyme SmaI and dephosphorylated. After electroporation of the E. coli DH10B strain, 400 clones were isolated and, of the latter, 330 were sequenced (290 in both orientations, 40 in the + or −0 orientation) at Genoscope France. The readings were collected in a single contig of 44.16 kb, the composition of which in bases is in conformity with that of the DNA of the phage, i.e. 69.3% G:C and 30.7% A:T (instead of D:T). All of the ORFs deduced from this contig were compared to different databases, which made it possible to annotate quite particularly 54 of them represented in Table 1 and quite particularly 14 of them (only taking account of statistically significant homologies with known bacterial or phage proteins) shown as “very significant” in Table 1.

| | | | | Very | SEQ ID No. and |
|---|---|---|---|---|---|
| PROTEIN | aa Number | Frame | Position | significant | ORF No. |
| DNA A chromosome replication | 134 | −2 | 661-1062 | | 14 |
| initiator protein | | | | | |
| DNA polymerase | 50 | 1 | 963-1112 | | 18 |
| Polyketide synthetase | 177 | −1 | 1308-1838 | | 26 |
| Betaglucosidase | 135 | −1 | 4698-5102 | | 68 |
| DNA helicase | 51 | 2 | 6280-6432 | X | 86 |
| Sigma factor | 203 | −3 | 6806-7414 | X | 92 |
| Ribonucleoprotein | 100 | 1 | 8064-8363 | | 105 |
| DNA-binding protein | 656 | −2 | 8320-10287 | | 109 |
| RNA-binding protein | 92 | 1 | 10299-10574 | | 134 |
| Replicase | 55 | −1 | 11052-11216 | | 142 |
| DNA-directed putative RNA Pol III | 72 | 3 | 11237-11452 | | 143 |
| broad sub-unit | | | | | |
| DNA topoisomerase I | 186 | −1 | 11613-12170 | | 148 |
| DNA packaging protein | 448 | −2 | 11956-13299 | X | 152 |
| Capsid protein | 109 | 1 | 13629-13955 | | 169 |
| Adenylosuccinate synthetase | 419 | −1 | 14235-15491 | X | 175 |
| Putative reverse transcriptase | 113 | 1 | 15132-15470 | | 187 |
| Transposase | 65 | 3 | 16799-16993 | | 208 |
| Exodeoxyribonuclease VIII | 347 | −2 | 17113-18153 | X | 211 |
| DNA helicase | 424 | 3 | 18962-20233 | X | 234 |
| DS RNA Adenosine deaminase | 172 | 3 | 20237-20752 | | 246 |
| rRNA Adenine N-6 | 63 | −2 | 20671-20859 | | 250 |
| methyltransferase | | | | | |
| Virulence protein E | 738 | 3 | 20921-23134 | X | 257 |
| RNA Polymerase II | 69 | −1 | 21444-21650 | | 264 |
| broad sub-unit | | | | | |
| Putative tRNA cleavage | 251 | −1 | 23316-24068 | | 286 |
| endonuclease | | | | | |
| Type 1 restriction enzyme (M | 60 | −1 | 24072-24251 | X | 298 |
| protein) | | | | | |
| Envelope protein | 385 | 3 | 25685-26839 | X | 316 |
| Guanylyl cyclase | 111 | −1 | 27183-27515 | | 332 |
| Uracyl-DNA glycosylase | 65 | −2 | 28063-28257 | | 342 |
| N-6 aminoadenine-N | 74 | 1 | 28239-28550 | | 347 |
| methyltransferase | | | | | |
| Inosine 5′monophosphate | 67 | −1 | 28401-28601 | | 348 |
| dehydrogenase | | | | | |
| Membrane protein | 377 | −1 | 28617-29747 | | 351 |
| Polymerase | 94 | −3 | 28871-29152 | | 355 |
| Transketolase | 82 | −1 | 29751-29996 | | 364 |
| Ribulose biphosphate carboxylase | 61 | 2 | 29893-30075 | | 365 |
| Tail absorption protein | 860 | 1 | 30105-32684 | | 369 |
| RNA-binding protein | 247 | 2 | 30118-30858 | | 370 |
| DNA Pol III gamma sub-unit | 103 | −2 | 31876-32184 | | 380 |
| M λ tail protein | 159 | 1 | 32889-33365 | | 392 |
| L λ tail protein | 509 | 3 | 33023-34549 | X | 395 |
| K λ tail protein | 271 | 2 | 30118-30858 | X | 406 |
| RNA Pol beta (fragment) | 84 | 3 | 35267-35518 | | 418 |
| I λ tail protein | 236 | 2 | 35458-36165 | | 422 |
| J λ tail protein | 1456 | 1 | 35598-39965 | X | 425 |
| DNA topoisomerase I | 79 | 2 | 36169-36405 | | 429 |
| Dedoxyadenine methylase | 74 | −3 | 36428-36649 | | 432 |
| RNA Polymerase II | 146 | 2 | 36646-37083 | | 433 |
| broad sub-unit | | | | | |
| DNA Polymerase I | 63 | −3 | 39089-39277 | | 454 |
| DNA gyrase | 101 | 3 | 39989-40291 | | 464 |
| sub-unit B | | | | | |
| Dioxygenase | 301 | −2 | 40156-41058 | | 466 |
| RNA guanylyltransferase | 190 | 1 | 41097-41666 | | 472 |
| Lysozyme | 381 | 3 | 41951-43093 | X | 484 |
| RNA binding protein | 129 | −2 | 42457-42843 | | 489 |
| Tranposase/exonuclease | 110 | 3 | 43097-43426 | | 494 |
| Phosphodiesterase | 58 | −2 | 43417-43590 | | 500 |

These are in particular proteins involved in the formation and assembly of the tail of bacteriophage λ·M, L, K, I and J tail protein, GP17 protein which plays a role in the DNA packaging in bacteriophage T4, an exonuclease which could be involved in the exclusion of the A-base, an RNA helicase, a sigma factor and a succinyladenylate synthetase.

The identification of genes coding for a sigma factor and a helicase leads to the conclusion that the transcription of the genome of S-2L and the replication of the cyanophage DNA probably required specific proteins encoded by the phage, the activity of which could depend on the D-base.

On the other hand, it seems very likely that the D-base is formed by semi-replicative modification. Between the two biosynthesis routes of dDTP formation described above, the identification of a succinyladenylate synthetase gene homologue called ddbA (deoxyribodiaminopurine biosynthetic gene A) leads to the conclusion that it is the second route which is probably taken during phage infection (FIG. 2).

Several tests have been carried out in order to determine the activity of the corresponding protein. The results suggest that the expression of ddbA allows restoration of the growth of a strain of E. coli expressing the yaaG gene of Bacillus subtilis in the presence of a high concentration of dG (10 mM). On the other hand, 2,6-diaminopurine becomes toxic (10 mM) to E. coli when it is in phosphorylated form (which has been tested in the same strain of E. coli expressing the yaaG gene of Bacillus subtilis i.e. MG1655 pSU yaaG) which makes it possible to have a screen in order to identify in vivo the complete biosynthesis route of the D-base.

However from now on complete identification is not necessary in order to obtain D-bases by the processes described above.

Another approach consists of systematically expressing the ORFs specifying all the possible genes of S-2L and combining the raw activities resulting from this expression in order to cause the route metabolites to appear in vitro. An inducible metabolic route producing dDTP will then be created in E. coli by assembly of the appropriate genes. The route thus created will be applied to synthetic precursors in order to generate deviant nucleotides by the base and the sugar.

The use of the D-base in the replication and transcription processes is systematically researched in the extracts of the bacteria expressing the phage ORFs.

The above results were obtained by means of the following operations. The ddbA gene was expressed in E. coli under the control of an inducible promoter and several tests were carried out in order to determine the activity of the corresponding protein. The results obtained show that the expression of ddbA allows restoration of the growth of E. coli in the presence of a high concentration of dGMP. On the other hand, 2,6-diaminopurine becomes toxic to E. coli when it is in phosphorylated form which makes it possible to have a screen in order to identify in vivo the complete biosynthesis route of the D-base. The ddbA gene was amplified using 100 pmol of each ngaattcaagctttcagcgacggtagcgggcatac and nnnnccatggtgaagaactgcaacctgatc oligonucleotide,100 ng of DNA of S-2L as matrix DNA, 200 mM of each of the dNTPs, 10 ml of Pfu polymerase buffer concentrated 10 times, 10% DMSO and 5U Pfu polymerase. The amplification cycles were: a 10-minute stage at 95° C., then 25 cycles of 30 seconds at 95° C., 30 seconds at 56° C.,2 minutes 20 seconds at 72° C. then a 10-minute stage at 72° C. The amplification product was then purified using the Jetsorb Kit (Genomed GmbH) then digested by the restriction enzymes NcoI and HindIII. After purification, the amplification product was inserted into plasmid pBAD24 (Guzman et al., 1995 J Bacteriol 177: 4121-4130) digested by the same restriction enzymes. The ddbA gene in this construction is expressed starting with the araBAD operon promoter which is inducible by arabinose.

The cyanophage S-2L bank is maintained in the E. Coli strain β2033 deposited on 24th Jan. 2001 at the Collection Nationale de Cultures de Microorganismes, Institut Pasteur, 25 rue du Dr Roux, 75724 PARIS Cedex 15, France, according to the provisions of the Budapest Treaty, and registered under serial number 1-2619.

Thanks to the work carried out by the inventors, the sequencing of the genome of S-2L makes it possible to alter, inhibit or diversify the synthesis of nucleic acids in vitro and in vivo.

1. Nucleotide sequence of cyanophage S-2L, characterized in that it corresponds to SEQ ID No. 1.

2. Nucleotide sequence of cyanophage S-2L, characterized in that it is chosen from:
a) a nucleotide sequence comprising at least 80% identity with SEQ ID No. 1;
b) a nucleotide sequence hybridizing under high stringency conditions with SEQ ID No. 1;
c) a nucleotide sequence which complements SEQ ID No. 1 or which complements a nucleotide sequence as defined in a), or b), or a nucleotide sequence of the corresponding RNA;
d) a nucleotide sequence of a representative fragment of SEQ ID No. 1, or of a representative fragment of a nucleotide sequence as defined in a), b) or c);
e) a nucleotide sequence comprising a sequence as defined in a), b), c) or d); and
f) a nucleotide sequence modified from a nucleotide sequence as defined in a), b), c), d) or e).

3. Nucleotide sequence according to claim 2, characterized in that it codes for a polypeptide chosen from:
a) the polypeptides of cyanophage S-2L of sequences SEQ ID No. 2 to SEQ ID No. 527;
b) preferably the polypeptides of sequence SEQ ID No. 14, 18, 26, 68, 86, 92, 105, 109, 134, 142, 143, 148, 152, 169, 175, 187, 208, 211, 234, 246, 250,257, 264, 286, 298, 316, 332, 342, 347, 348, 351, 355, 364, 365, 369, 370, 392, 395, 406, 418, 422, 425, 429, 432, 433, 454, 464, 466, 472, 484, 489, 494, 500;
c) preferably also the polypeptides of sequence SEQ ID No. 86, 92, 152, 175, 234, 257, 298, 316, 395, 406, 425, 484;
d) the polypeptides having at least 80% preferably 85%, 90%, 95% and 98% identity with a polypeptide from a), b), c);
e) the biologically active fragments of the polypeptides from a), b), c), d)
f) the polypeptides modified from a), b), c), d), e).

4. Nucleotide sequence characterized in that it comprises a nucleotide sequence chosen from:
a) a nucleotide sequence according to claim 3;
b) a nucleotide sequence comprising at least 80% identity with a nucleotide sequence according to claim 3;
c) a nucleotide sequence hybridizing under high stringency conditions with a nucleotide sequence according to claim 3;
d) a complementary or RNA nucleotide sequence corresponding to a sequence as defined in a), b) or c);
e) a nucleotide sequence fragment representative of a sequence as defined in a), b), c) or d); and
f) a nucleotide sequence modified from a sequence as defined in a), b), c), d) or e).

5. Polypeptide encoded by a nucleotide sequence according to one of claims 2 to 4.

6. Polypeptide according to claim 5, characterized in that it is chosen from the peptides of sequence SEQ ID No. 2 to 527, preferably from the sequences SEQ ID No. 14, 18, 26, 68, 86, 92, 105, 109, 134, 142, 143, 148, 152, 169, 175, 187, 208, 211, 234, 246, 250, 257, 264, 286, 298, 316, 332, 342, 347, 348, 351, 355, 364, 365, 369, 370, 392, 395, 406, 418, 422, 425, 429, 432, 433, 454, 464, 466, 472, 484, 489, 494, 500;

7. Polypeptide according to claim 5 or 6, characterized in that it is chosen from the sequences SEQ ID No. 86, 92, 152, 175, 234, 257, 298, 316, 395, 406, 425, 484;

8. Polypeptide characterized in that it comprises a polypeptide chosen from:
a) a polypeptide according to one of claims 5 to 7;
b) a polypeptide having at least 80% identity with a polypeptide according to one of claims 5 to 7;
c) a fragment of at least 5 amino acids of a polypeptide according to one of claims 5 to 7, or as defined in b);
d) a biologically active fragment of a polypeptide according to one of claims 5 to 7, or as defined in b) or c); and
e) a polypeptide modified from a polypeptide according to one of claims 5 to 7, or as defined in b), c) or d).

9. Nucleotide sequence according to one of claims 2 to 4, characterized in that it codes for a polypeptide of cyanophage S-2L, involved in the biosynthesis of nucleotides, purines, pyrimidines or nucleosides or for one of its representative fragments.

10. Nucleotide sequence according to one of claims 2 to 4, characterized in that it codes for a polypeptide of cyanophage S-2L, involved in the biosynthesis of D-bases or for one of its representative fragments.

11. Nucleotide sequence according to claim 10, characterized in that it codes for a peptide of sequence SEQ ID No. 175.

12. Nucleotide sequence according to one of claims 2 to 4, characterized in that it codes for a polypeptide of cyanophage S-2L, involved in the replication process, or for one of its representative fragments, preferably a peptide of sequence SEQ ID No. 14, 18, 142, 355, 429, 454 (DNA polymerase, topoisomerase activity).

13. Nucleotide sequence according to one of claims 2 to 4, characterized in that it codes for a polypeptide of cyanophage S-2L, involved in the transcription process, or for one of its representative fragments, preferably a peptide of sequence SEQ ID No. 92, 143, 187, 234, also preferably SEQ ID No. 92.

14. Nucleotide sequence according to one of claims 2 to 4, characterized in that it codes for an envelope polypeptide of cyanophage S-2L, or one of its representative fragments, preferably a peptide of sequence SEQ ID No. 169, 316, 351, 392, 395, 406, 422, 425, in particular a peptide of sequence SEQ ID No. 395, 406, 425.

15. Nucleotide sequence according to one of claims 2 to 4, characterized in that it codes for a polypeptide of cyanophage S-2L involved in the rerouting of the cell machinery or one of its representative fragments.

16. Nucleotide sequence according to one of claims 2 to 4, characterized in that it codes for a polypeptide of cyanophage S-2L involved in virulence or one of its representative fragments, preferably a peptide of sequence SEQ ID No. 257.

17. Polypeptide according to one of claims 5 to 8, characterized in that it is a polypeptide of cyanophage S-2L involved in the biosynthesis of nucleotides, purines, pyrimidines or nucleosides.

18. Polypeptide according to one of claims 5 to 8, characterized in that it is a polypeptide of cyanophage S-2L involved in the biosynthesis of D-bases, preferably a peptide of sequence SEQ ID No. 175 or a representative fragment.

19. Polypeptide according to one of claims 5 to 8, characterized in that it is a polypeptide of cyanophage S-2L involved in the replication process, preferably a peptide of sequence SEQ ID No. 14, 18, 142, 355, 429, 454 or a representative fragment.

20. Polypeptide according to one of claims 5 to 8, characterized in that it is a polypeptide of cyanophage S-2L, involved in the transcription process, preferably a peptide of sequence SEQ ID No. 92, 143, 187 or a representative fragment.

21. Polypeptide according to one of claims 5 to 8, characterized in that it is an envelope polypeptide of cyanophage S-2L, preferably a peptide of sequence SEQ ID No. 169, 316, 351, 392, 395, 406, 422, 425 or a representative fragment.

22. Polypeptide according to one of claims 5 to 8, characterized in that it is a polypeptide of cyanophage S-2L involved in intermediate metabolism, or a representative fragment.

23. Polypeptide according to one of claims 5 to 8, characterized in that it is a polypeptide of cyanophage S-2L involved in the process of rerouting the cell machinery or one of its fragments.

24. DNA chip or filter, characterized in that it contains at least one nucleotide sequence according to any one of claims 2 to 4.

25. Cloning and/or expression vector, characterized in that it contains a nucleotide sequence according to one of claims 3 or 4 or 9 to 16.

26. Cloning and/or expression vector according to claim 25, characterized in that it contains a nucleotide sequence according to claim 10 or 11, in particular coding for a protein involved in the sythesis of D-bases.

27. Cloning and/or expression vector according to claim 25, characterized in that it contains a nucleotide sequence according to claim 11 or 12, in particular coding for a polymerase capable of polymerizing D-bases.

28. Host cell, characterized in that it is transformed by a vector according to one of claims 25 to 27.

29. Host cell according to claim 28, characterized in that it is a bacterium.

30. Process for preparing a polypeptide of interest, characterized in that a cell transformed by a vector according to one of claims 25 to 27 is cultured under conditions allowing the expression of said polypeptide, and in that said recombinant polypeptide is recovered.

31. Process according to claim 30, characterized in that the polypeptide of interest is a protein involved in the metabolism of D-bases, in particular succinyladenylate synthetase.

32. Process according to claim, characterized in that the polypeptide of interest is a polymerase of cyanophages S-2L, capable of polymerizing D-bases.

33. Recombinant polypeptide capable of being obtained by a process according to claim 30.

34. Monoclonal or polyclonal antibody, its fragments, or chimeric antibody, characterized in that it is capable of specifically recognizing a polypeptide according to one of claims 5 to 8 or 17 to 23.

35. Antibody according to claim 34, characterized in that it is a marked antibody.

36. Protein chip characterized in that it contains at least one polypeptide according to one of claims 5 to 8 or 17 to 23, or at least one antibody according to one of claims 34 or 35, immobilized on the support of said chip.

37. Process for detection and/or identification of cyanophage S-2L or of a related phage in a biological sample, characterized in that it uses a nucleotide sequence according to one of claims 3 to 4 or 9 to 16.

38. Process for obtaining D-bases and/or polynucleotides of interest comprising at least one D-base, comprising the culture of a microorganism containing at least one nucleotide sequence of cyanophage S-2L coding for at least one polypeptide involved in the synthesis of D-bases, under appropriate conditions for the development of the vector and the synthesis of D-bases and/or said polynucleotides of interest.

39. Process according to claim 38 comprising:
the addition to a medium comprising the substrates required for obtaining D-bases, of an extract or mixture of extracts of recombinant bacteria expressing at least one gene of cyanophage S-2L involved in the synthesis of D-bases
if appropriate the extraction of the polynucleotides of interest.

40. Process according to claim 38 comprising:
the preparation of at least one DNA sequence coding for a polypeptide capable of provoking the synthesis of at least one D-base in a host microorganism
the cloning of said coding sequence in a vector capable of being transferred into and replicating in said host microorganism, this vector comprising the elements necessary for the expression of said coding sequence
the transfer of the vector comprising said coding sequence into a microorganism capable of producing the enzymes of the D-base synthesis directed by said coding sequence
the culture of the microorganism under appropriate conditions for the development of the vector and the synthesis of the D-bases
if appropriate the extraction of D-bases and/or of said polynucleotides of interest.

41. Process for obtaining D-bases and/or polynucleotides of interest comprising at least one D-base, said process comprising:
the addition, to a medium comprising the substrates required for obtaining D-bases, of the expression product of at least one gene of cyanophage S-2L involved in the synthesis of D-bases, in order to produce D-bases and/or polynucleotides of interest comprising at least one D-base
the extraction of the D-bases and/or said polynucleotides of interest.

42. Process for obtaining polynucleotides of interest comprising at least one D-base, said process comprising the culture of a microorganism containing at least one nucleotide sequence of cyanophage S-2L coding for at least one polypeptide involved in the extension of said polynucleotides with incorporation of D-bases, DNA polymerase in particular, in appropriate conditions for the development of the vector and the extension of said polynucleotides.

43. Process according to any one of claims 38 to 42 characterized in that the gene involved in the synthesis of the D-bases is the gene of succinyladenylate synthetase.

44. Process according to any one of claims 38 to 42 characterized in that it comprises a stage of amplification, in the presence of cyanophage D polymerase and appropriate primers, of polynucleotides comprising at least one D-base.

45. Process for selection of compounds capable of stimulating or inhibiting the synthesis of D-bases and/or polynucleotides of interest incorporating at least one D-base, comprising the addition to the synthesis medium of the tested compound and comparison of the synthesis in the presence and in the absence of said compound.

46. Use of cyanophage S-2L for obtaining DNA polymerase or RNA polymerase involved in the metabolism of the D-bases.

47. Use of cyanophage S-2L for the production of nucleotides of interest not obtained naturally comprising at least one D-base, dDMP and dDTP.

48. Strain of cyanophage S-2L deposited at the CNCM No. 1-2619 characterized in that it contains at least one nucleotide sequence according to claims 1 to 4.

49. Process for producing a gene bank of cyanophage S-2L characterized in that the process comprises the following stages:
a) Culture of purified cyanophages S-2L from the species Synechococcus,
b) Fragmentation of the DNA extracted by the sonication technique,
c) Cloning of the Shotgun bank obtained in an E. coli vector, and
d) Sequencing of the clones until the genome is completely covered.

50. Gene bank of cyanophage S-2L deposited on 24th Jan. 2001 at the C.N.C.M. according to the provisions of the Budapest Treaty, and registered under accession number 1-2619 obtained by a process according to claim 49.

51. Plasmids contained in recombinant bacteria deposited at the C.N.C.M. under reference number 1-2619.

52. Recombinant bacteria such as deposited at the C.N.C.M. under reference number 1-2619.
